(19) United States
(12) Patent Application Publication
(10) Pub. No.: US 2006/0068047 A1
(43) Pub. Date: Mar. 30, 2006

(54) Adsorbent for endocrine disruptors and foods and drinks containing the same

(75) Inventors: Kenji Ohishi, Tokyo (JP); Haruji Sawada, Tokyo (JP); Wakae Yokoi, Tokyo (JP); Yasuto Yoshida, Tokyo (JP); Tsunekazu Watanabe, Tokyo (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: KABUSHIKI KAISHA YAKULT HONSHA, Tokyo (JP)

(21) Appl. No.: 11/246,129

(22) Filed: Oct. 11, 2005

Related U.S. Application Data
(62) Division of application No. 10/276,699, filed on Nov. 18, 2002, now abandoned.

(30) Foreign Application Priority Data
May 16, 2000 (JP) 2000-143025

(51) Int. Cl. A61K 35/74

(52) U.S. Cl. 424/780

(57) ABSTRACT

An adsorbent for endocrine disruptors which contains as the active ingredient viable or dead cells of intestinal microorganisms or constituents thereof. When the adsorbent for endocrine disruptors and foods and drinks containing the same are orally taken either daily or simultaneously with endocrine disruptors typified by bisphenols, alkylphenols and triazines and foods and drinks contaminated therewith, the absorption of these endocrine disruptors into the body can be inhibited and the discharge from the body of the endocrine disruptors, which have been once incorporated into the body, can be promoted.

### TECHNICAL FIELD

The present invention relates to an adsorbent for endocrine-disrupting chemicals which can adsorb and eliminate orally taken endocrine-disrupting chemicals and, more particularly, to an adsorbent for endocrine-disrupting chemicals and food containing the same, which not only can adsorb endocrine-disrupting chemicals in the enteric lumen of human or animals to suppress in vivo absorption of such chemicals, but also can accelerate excretion of such in vivo-absorbed chemicals.

### BACKGROUND ART

Endocrine-disrupting chemicals (or environmental hormones) are noted as materials which cause a serious social problem since organotin compounds, which have been coated onto the bottoms of ships for preventing attachment of shellfish over a long time, were reported to cause abnormalities in the generative organs of shellfish (Gibbs et al., J. Mar. Biol. Assoc. UK, 66, 767, 1986). The endocrine system of organisms is a system essential for controlling the evolution of individuals, development of generative organs, and homeostatic maintenance of organs by the action of various hormones. Endocrine-disrupting chemicals are substances which disrupt the balance of the endocrine system and cause abnormal growth and reproduction of animals living in lakes and swamps polluted by domestic waste water.

Humans and animals are said to absorb endocrine-disrupting chemicals through the digestive tract, lungs, and skin. However, absorption of endocrine-disrupting chemicals by humans through air, water, or soil is comparatively small in a normal living environment. Humans are considered to be polluted by such chemicals mainly through daily food intake (meat and fish). In particular, triazine herbicides such as “Atrazine” (shown by the following formula (I)) and “Simazine”, as well as other agricultural chemicals such as “Malathion” which are currently used are reported to be dangerous because they contaminate biological organs. These orally-taken endocrine-disrupting chemicals are absorbed from the intestinal tract and accumulated in the liver and adipose tissues.

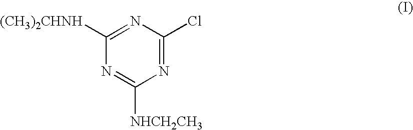

On the other hand, bisphenol A eluted from polycarbonate resins and the like used for various food containers (Krishnan et al., Endocrinology, 132, 2279 (1993), Olea et al., Environ. Health Perspect., 104, 298 (1996)) and 4-nonylphenol (Soto et al., Environ. Health Perspect., 92, 167 (1991), Nimrod et al., Crit. Rev. Toxicol., 26, 335 (1996)) binds to estrogen receptors, female hormones, and exhibit a significant effect on humans. These chemicals are thus the source of a serious social problem.

Bisphenol A is a chemical compound having the following formula (II).

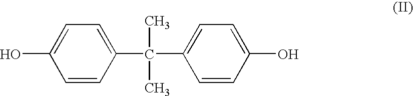

The compound is mainly used as a raw material for polycarbonate resin and epoxy resin, as a raw material for other resins such as phenol resin, plastic polyester, polysulfone, acrylic resin, etc., as a stabilizer for polyvinyl chloride, and as an antioxidant. Production of bisphenol A in Japan amounted to about 250,000 tons in 1996, of which 40,000 tons were used for food applications.

Because polycarbonate resin made from bisphenol A excels in heat resistance and impact resistance, the resin is used in instruments used at high temperatures, such as coffee drippers, tableware for infants and school children, feeding bottles, and the like. Polycarbonate products not only contain unreacted bisphenol A, but also easily release bisphenol A from the polymer at high temperature.

In addition, many steel or aluminum cans have epoxy resin or vinyl chloride resin coated over the inner surface. Epoxy resin contains bisphenol A as a raw material and vinyl chloride resin also contains bisphenol A as a stabilizer. The possibility that the bisphenol A remaining in the coating of the steel or aluminum cans may be dissolved from the coating into food has been pointed out (Brotons et. al, Environ. Health Perspect., 103, 608 (1995)).

In addition, bisphenol A is included in polycarbonate plastic used as a dental sealant when treating cavities or as a tooth coating agent applied to children (Keith et al. “Environmental Endocrine Disruptors” Willey-Inteuscience, New York, p 1232 (1997)). Risk of bisphenol A elusion from such a dental material when polymerization is imperfect or when heat is applied during high-pressure steam sterilization has been pointed out (Krishnan et al., Endocrinology, 132, 2279 (1993)).

4-Nonylphenol of the following formula (III) is used as a nonionic surfactant in the form of nonylphenol ethoxylate. The annual production amounts to about 20,000 tons. Due to low foaming properties, nonylphenol ethoxylate is used mainly as an industrial detergent or dispersant in the textile industry, paper industry, metal industry, and pesticide industry in Japan. Sewage treatment effluent containing 4-nonylphenol flowing into rivers and ocean pollutes fish and other animals.

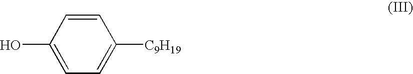

In this manner, humans are exposed to the risk of various endocrine-disrupting chemicals such as bisphenol A, 4-nonylphenol, and atrazine in everyday life. Therefore, urgent development of a material for preventing humans from being polluted by these chemicals and a method of removing these chemicals is desired. Endocrine-disrupting chemicals orally taken and absorbed from the intestinal tract reach all body organs through the blood flow and, as a result, may induce various adverse effects such as imbalance in the endocrine system. If a substance that can adsorb endocrine-disrupting chemicals in the intestinal tract is discovered, such a substance may be used for suppressing in vivo absorption of endocrine-disrupting chemicals and accelerating excretion of the endocrine-disrupting chemicals. Thus, the substance can protect biological organs from being disrupted by the endocrine-disrupting chemicals according to the above mechanism.

In this relation, Morita et al. reported that rice-bran fiber, spinach fiber, chlorella, and spirulina accelerate excretion of dioxines and some polychlorinatedbiphenyls (PCB), which are endocrine-disrupting chemicals (Morita et al., Jpn. J. Toxicol. Environ. Health, 43, 42-47 (1997)).

However, there has been no report as yet about the effect of microorganisms that are present in the intestinal tract to adsorb endocrine-disrupting chemicals and suppress in vivo absorption of such endocrine-disrupting chemicals by humans and animals.

The present invention has been achieved in view of this situation and has an object of providing a medicine which is taken orally in daily life together with endocrine-disrupting chemicals such as bisphenol A, 4-nonylphenol, and atrazine dissolved from tableware or food polluted by such endocrine-disrupting chemicals, and can suppress in vivo absorption of endocrine-disrupting chemicals and accelerate excretion of such chemicals once-absorbed.

### DISCLOSURE OF THE INVENTION

The inventor of the present invention has conducted extensive studies to discover a substance which can adsorb endocrine-disrupting chemicals, particularly chemical compounds such as bisphenols, alkylphenols, and triazines. As a result, the inventor has found that cells of various enterobacteria or the constituents thereof can effectively adsorb these substances. This finding has led to the completion of the present invention.

Specifically, the present invention provides an adsorbent for endocrine-disrupting chemicals comprising live bacteria or dead bacteria of the enterobacteria or a constituent thereof as an active component.

The present invention also provides food comprising the above adsorbent for endocrine-disrupting chemicals.

The present invention further provides a method of suppressing absorption of endocrine-disrupting chemicals by the human body or a method of accelerating excretion of endocrine-disrupting chemicals from the human body, comprising administering the above adsorbent for endocrine-disrupting chemicals to humans.

### BRIEF DESCRIPTION OF THE DRAWINGS

FIG. 1 shows the relation between the enterobacteria concentration and the amount of bisphenol A adsorbed by the enterobacteria when only the enterobacteria are present in the reaction mixture.

FIG. 2 shows the relation among the enterobacteria concentration, the amount of residual bisphenol A in the supernatant of the reaction mixture, and the amount of adsorbed bisphenol A by the enterobacteria.

FIG. 3 shows the relation between the enterobacteria concentration and the amount of bisphenol A adsorbed when enterobacteria are present in diet.

FIG. 4 shows the relation between the diet concentration and the amount of bisphenol A adsorbed by enterobacteria.

FIG. 5 shows the relation between the pH of the reaction system and the amount of bisphenol A adsorbed by the enterobacteria.

FIG. 6 shows the relation between the reaction time and the amount of bisphenol A adsorbed by the enterobacteria.

FIG. 7 shows the relation between the types of enterobacteria and the amount of bisphenol A adsorbed by the enterobacteria.

FIG. 8 shows the relation between the types of cellulose and the amount of bisphenol A adsorbed by the enterobacteria.

FIG. 9 shows the relation between the types of enterobacteria and the amount of 4-nonylphenol adsorbed by the enterobacteria.

FIG. 10 shows the relation between the types of enterobacteria and the amount of atrazine adsorbed by the enterobacteria.

FIG. 11 shows the relation among the types of enterobacteria, the amount of the enterobacteria administered, and the total amount of bisphenol A excreted as excrements.

FIG. 12 shows the relation among the types of enterobacteria, the amount of enterobacteria administered, and the amount of dry excrements.

FIG. 13 shows the relation among the types of enterobacteria, the amount of enterobacteria administered, and the number of live bacteria in caecum contents out of the administered bacteria.

FIG. 14 shows the relation between the amount of enterobacteria administered and the bisphenol A concentration in dry excrements.

### DETAILED DESCRIPTION OF THE INVENTION AND PREFERRED EMBODIMENT

Enterobacteria used as the adsorbent for endocrine-disrupting chemicals of the present invention are microorganisms known as bacterial strains used for the production of food (lactic acid bacteria beverage, yogurt, etc.) and are extremely safe for humans. Enterobacteria of the present invention include not only bacteria present in the intestine, but also bacteria which are taken in food and drinks, and stay in the intestine for a long time.

As examples of enterobacteria, microorganisms belonging to Genuses Lactobacillus, Bifidobacterium, Lactococcus, Streptococcus, Enterococcus, Weissella, Leuconostoc, Tetragenococcus, Propionibacterium, Bacteroides, Clostridium, Eubacterium, Prevotella, Pediococcus, and Megasphaera can be given.

All microorganisms belonging to the above genuses are readily available. Specific examples of particularly preferable microorganisms are as follows.
Lactobacillus casei YIT 9029 FERM BP-1366 (May 1, 1981).
Lactobacillus acidophilus YIT 0168 FERM BP-7536 (Dec. 16, 1981).
Bifidobacterium breve YIT 4065 FERM BP-6223 (Feb. 29, 1996).
Bifidobacterium bifidum YIT 4007 FERM BP-791 (May 1, 1981).
Lactococcus lactis YIT 2027 FERM BP-6224 (Feb. 10, 1997).
Streptococcus thermophilus YIT 2001 FERM BP-7538 (Jan. 31, 2001).
Streptococcus thermophilus YIT 2021 FERM BP-7537 (Nov. 1, 1996).

The above microorganisms are deposited with the International Patent Organism Depositary National Institute of Advanced Industrial Science and Technology*, AIST Tsukuba Central 6, 1-1, Higashi 1-Chome Tsukuba-shi, Ibaraki-ken 305-8566 Japan (*present name).
Enterococcus faecium YIT 2039
Weissella confusa YIT 0233
Leuconostoc lactis YIT 3001
Pediococcus acidilactici YIT 3025
Propionibacterium acidipropionici YIT 3501
Megasphaera elsdenii YIT 6063

Live bacteria of enterobacteria of the present invention can be obtained by culturing these bacteria in a composite culture medium containing yeast extract and polypeptone. The composition of a preferable composite culture medium is given below. The above-described microorganisms may be used either individually or in combination of two or more as the enterobacteria of the present invention. Two or more microorganisms belonging to different species or genuses may be used in combination.

(Composite Culture Medium Composition)

Modified GAM broth (manufactured by NISSUI PHARMACEUTICAL CO., LTD.) 41.7 g
D-(+)-glucose (manufactured by Wako Pure Chemicals Industries, Ltd.) 10.0 g
Polyoxyethylene (20) Sorbitan monooleate 1.0 g
(manufactured by Wako Pure Chemicals Industries, Ltd.) Deionized water 1.0 liter (pH 7.3)

Heated cells (dead bacteria) of the above enterobacteria can be obtained, for example, by heating the above live bacteria at a temperature between 80-120° C. for about 15 to 30 minutes.

On the other hand, the constituent of enterobacteria of the present invention can be obtained by a conventional method. Such a constituent may be a product obtained by processing the enterobacteria by a suitable means not adversely affecting the effect of the present invention, and may include protoplast fractions obtained by removing cell walls by treating the above cells with a cell wall digesting enzyme, soluble fractions obtained by treating the above cells with a cell wall digesting enzyme, cytoplasm membrane fractions obtained by treating the protoplast fractions with an organic solvent, pulverized products of the above cells, cell wall fractions obtained by treating the pulverized products of the above cells with a nuclease and proteinase.

Live bacteria, heat treated bacteria (dead bacteria), and constituents of the enterobacteria thus obtained (hereinafter referred to as “enterobacteria cells”) are used as is, or in combination with known pharmaceutically acceptable carriers, as the adsorbent for endocrine-disrupting chemicals of the present invention.

Although a dose required for the adsorbent for endocrine-disrupting chemicals to exhibit its effect in the intestinal tract differs according to the type of cells, a dose in the range of 10 mg to 30 g/day, and preferably 1 to 5 g/day, is generally sufficient. Therefore, the adsorbent for endocrine-disrupting chemicals of the present invention may be formulated into a preparation appropriate for providing this amount of dosage.

Moreover, the adsorbent for endocrine-disrupting chemicals of the present invention may be incorporated into various food and beverages. As examples of such food and drinks, fermented milk, fruit juice, soup, rice cracker, and cookie can be given. Although there are no limitations to the amount of adsorbent for endocrine-disrupting chemicals to be added to these foods and drinks, such an amount should be sufficient for a person to ultimately receive the above-defined dosage.

The adsorbent for endocrine-disrupting chemicals and food and drinks containing the adsorbent of the present invention thus obtained can adsorb various endocrine-disrupting chemicals, for example, bisphenols such as bisphenol A, alkylphenols such as 4-nonylphenol, and triazines such as atrazine, thereby preventing these chemicals from being absorbed in vivo, and accelerating excretion of these chemicals. The adsorbent for endocrine-disrupting chemicals thus can protect the endocrine system from being disrupted by these chemicals.

### EXAMPLES

The present invention will be described in more detail by way of Examples which should not be construed as limiting the present invention. In the following examples, the amount of endocrine-disrupting chemicals adsorbed in bacteria and the amount excreted by animals were measured according to the following method.

(1) Bisphenol A Adsorption Effect of Microorganisms

(a) Preparation of Sample

One platinum loop of cells stored in a dispersion medium was inoculated into 10 ml of the above composite culture medium in a test tube (15 ml) and incubated at 37° C. After 24 hours, the content of one test tube was placed in a conical flask (500 ml) containing 240 ml of the same medium, and the mixture was allowed to stand for 16 hours at 37° C. After culturing, the culture broth was separated into cells and supernatant liquid by centrifugation with cooling under the following conditions. The cells were washed twice with a phosphate buffer (a mixture of a 20 mM aqueous solution of potassium dihydrogenphosphate and a 20 mM aqueous solution of dipotassium hydrogenphosphate, adjusted to pH 7.0). The washed cells or heat treated bacteria obtained by treating the cells at 80° C. for 30 minutes were suspended in a phosphate buffer for use in the adsorption test.

(Conditions of Cooling Centrifugation)

Cooling centrifugal separator: RS-18III (manufactured by Tomy Seiko Co., Ltd.)
Rotor: TA-18BH (manufactured by Tomy Seiko Co., Ltd.)
Rotation (gravitational acceleration): 8,000 rpm (12,000×g)
Centrifugation time: 20 minutes
Temperature: 4° C.
   (b) Preparation of Bisphenol A Sample

100 mg of bisphenol A (manufactured by Tokyo Kasei Kogyo Co., Ltd.) was weighed in a 10 ml measuring flask using an electronic balance and dissolved in ethanol (manufactured by Wako Pure Chemicals Industries, Ltd., special grade) to obtain a final volume of precisely 10 ml. 1 ml of this solution was transferred to another measuring flask (50 ml) using a transfer pipette and diluted with ethanol to precisely 50 ml.

Bisphenol B of the following chemical formula (IV) was used as an internal standard substance. 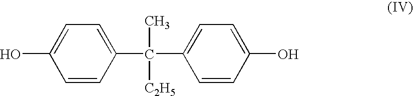
(c) Bisphenol A Adsorption by Microorganisms and Measurement of Residual Amount of Bisphenol A

0.2 ml of the ethanol solution of bisphenol A obtained in (b) above was added to the glass test tube containing 3.8 ml of the suspension of live bacteria or heat treated bacteria described in (a) above. The mixture was thoroughly stirred and allowed to stand in a water bath at 37° C. After 60 minutes, the cells were precipitated by centrifugation and 0.95 ml of the supernatant liquid was collected in a separate glass tube. 0.05 ml of ethanol solution (bisphenol B internal standard substance) and 1 ml of dichloromethane were added, and the tube was vigorously agitated by a shaker. After 20 minutes, the dichloromethane layer was separated from the aqueous layer by centrifugation, and 0.2 ml of the dichloromethane layer was collected into another glass tube. The dichloromethane was evaporated to dryness and the residue was allowed to stand in a desiccator overnight. A sample obtained by carrying out the above procedure in the absence of microorganisms was used as a negative control.

Next, 0.16 ml of dichloromethane was added to the residue. After redissolving the bisphenol A, N,O-bis (trimethylsilyl)-trifluoroacetamide (BSTFA) was added to convert the phenolic group into a trimethylsilyl group. The mixture was allowed to stand for one hour at room temperature. After the reaction, bisphenol A was quantitatively analyzed by gas chromatography (GC) under the following conditions.

(Gas Chromatography Measuring Conditions)

Gas chromatography apparatus: GC-14A (manufactured by Shimadzu Corp.)
Capillary column NEUTRABOND-1 (manufactured by GL Sciences Inc., 25 m×0.25 mm; df=0.4 μm)
Column temperature: 100° C.->300° C., 10° C./min
Carrier gas: helium
Injector: Split, T=310° C.
Detector: FID, T=310° C.
Injection volume: 1 μl
Analytical time: 20 minutes
   (d) Calculation of Amount of Bisphenol A Adsorbed by Microorganisms from the Residual Amount of Bisphenol A

The amount of bisphenol A adsorbed by microorganisms was calculated from the amount of bisphenol A not adsorbed by microorganisms determined in (c) above according to the following equation.
Y=X−(SA×CB/SB×CA)×X
Y: The amount of bisphenol A adsorbed by microorganisms
X: The amount of bisphenol A added to the reaction solution
CA: The peak area of bisphenol A contained in the reaction solution in the absence of microorganisms
CB: The peak area of bisphenol B corresponding to CA
SA: The peak area of bisphenol A not adsorbed by microorganisms
SB: The peak area of bisphenol B corresponding to SA
   (e) Bisphenol A Adsorption by Microorganisms and Measurement of Adsorbed Amount of Bisphenol A

0.2 ml of the ethanol solution of bisphenol A obtained in (b) above was added to the glass test tube containing 3.8 ml of the suspension of live bacteria or heat treated bacteria described in (a) above. The mixture was thoroughly stirred and allowed to stand in a water bath at 37° C. After 60 minutes, bacteria were precipitated by centrifugation and the supernatant liquid was placed in a separate glass tube. The bacteria were washed twice with a phosphate buffer (a mixture of a 20 mM aqueous solution of potassium dihydrogenphosphate and a 20 mM aqueous solution of dipotassium hydrogenphosphate, adjusted to pH 7.0). 20 μg of bisphenol A-d16 (the surrogate substance) and 2 ml of dichloromethane were added to the bacteria pellet and the supernatant, and the mixture was vigorously stirred (200 stroke/min). After 20 minutes, 0.2 ml of supernatant liquid obtained by centrifugation was collected in a separate glass tube. The dichloromethane was evaporated to dryness and the residue was allowed to stand in a desiccator overnight.

Next, 0.26 ml of dichloromethane was added to the residue. After redissolving the bisphenol A, BSTFA was added to convert the phenolic group into a trimethylsilyl group. The mixture was allowed to stand for one hour at room temperature. After the trimethylsilylation (TMS) reaction, 0.1 ml of a solution of phenanthrene-d10 (the internal standard substance) in dichloromethane (10 μg/ml) was added and bisphenol A was quantitatively analyzed by gas chromatography/mass spectrometer (GC/MS) under the following conditions.

(Gas Chromatography Measuring Conditions)

Gas chromatograph apparatus: GC-17A (manufactured by Shimadzu Corp.)
Capillary column HP-5 (manufactured by Agilent Inc., 30 m×0.25 mm; df=0.25 mm)
Liquid phase: 5% phenyl methyl silicone
Column temperature: 60° C. (1 minute)->280° C. (5 minutes), 10° C./min
Temperature at the inlet port: 280° C.
Method of injection: Splitless method (purge after one minute)
Injection volume: 1 μl
Carrier gas: High purity helium gas, linear velocity=44 cm/sec
Interface temperature: 280° C.
Sampling time: 1 minute
Analytical time: 28 minutes
   (Mass Spectrometry Measurement Conditions)
Mass spectrometer: QP5000 (manufactured by Shimadzu Corp.)
Ionization method: EI (70 eV)
Detector voltage: 1.5 kV
Detection mode: SIM
Sampling rate: 0.2 second
   (Measured Ions)

The objects of measurement and measured ions of the standard substances in the surrogate are shown in Table 1.

TABLE 1

| Object of measurement | Measured ions |
|---|---|
| Bisphenol A * | 357, 372 |
| Bisphenol A-d16 * | 368, 371, 386 |
| Phenanthrene A-d10 | 188 |

* TMS (trimethylsilylated) derivative

(f) Calculation of the Amount of Bisphenol A Adsorbed by Microorganisms

The detected amount of bisphenol A was determined using a calibration curve from the ratio of the peak area of the trimethylsilylation (TMS) product of bisphenol A and that of the surrogate substance determined by the GC/MS measurement. The amount of bisphenol A was then calculated from the detected amount, the amount of sample injected into GC/MS apparatus, the concentration magnification, etc. using the following formula.
Y=D×(L×1000/I)
Y: The amount of bisphenol A adsorbed by microorganisms (μg)
D: Amount detected by the GC/MS (μg)
L: Final liquid amount of the sample (ml)
I: Amount sample injected to GC/MS (μl)
   (2) 4-Nonylphenol Adsorption Effect of Various Microorganisms
   (a) Preparation of Sample

Samples were prepared in the same manner as in (1)(a) above.

(b) Preparation of Sample for 4-Nonylphenol

100 mg of 4-nonylphenol (manufactured by GL Sciences Inc.) was weighed in a 10 ml measuring flask using an electronic balance and dissolved in ethanol to obtain a final volume of precisely 10 ml. 3 ml of this solution was transferred to another measuring flask (50 ml) using a transfer pipette and diluted with ethanol to precisely 50 ml.

Bisphenol B described above was used as an internal standard substance.

(c) 4-Nonylphenol Adsorption by Microorganisms and Measurement of Residual Amount of 4-Nonylphenol

0.2 ml of the ethanol solution of 4-nonylphenol obtained in (b) above was added to the glass test tube containing 3.8 ml of the suspension of live bacteria or heat treated bacteria described in (a) above. The mixture was thoroughly stirred and allowed to stand in a water bath at 37° C. After 60 minutes, the cells were precipitated by centrifugation and 0.95 ml of the supernatant liquid was collected in a separate glass tube. 0.05 ml of ethanol solution (bisphenol B internal standard substance) and 1 ml of dichloromethane were added, and the tube was vigorously agitated by a shaker. After 20 minutes, the dichloromethane layer was separated from the aqueous layer by centrifugation, and 0.2 ml of the dichloromethane layer was collected into another glass tube. The dichloromethane was evaporated to dryness and the residue was allowed to stand in a desiccator overnight. A sample obtained by carrying out the above procedure in the absence of microorganisms was used as a negative control.

Next, 0.16 ml of dichloromethane was added to the residue. After redissolving the 4-nonylphenol, N,O-bis (trimethylsilyl) trifluoroacetamide (BSTFA) was added to convert the phenolic group into a silyl group. The mixture was allowed to stand for one hour at room temperature. After the trimethylsilylation reaction, 4-nonylphenol was quantitatively analyzed by gas chromatography (GC) under the same conditions as those applied to bisphenol A.

(d) Calculation of the Amount of 4-Nonylphenol Adsorption by Microorganisms

The amount of 4-nonylphenol adsorbed by microorganisms was calculated from the amount of 4-nonylphenol not absorbed by microorganisms determined in (c) above according to the following equation.
Y=X−(SA×CB/SB×CA)×X
Y: The amount of 4-nonylphenol adsorbed by microorganisms
X: The amount of 4-nonylphenol added to the reaction solution
CA: The peak area of 4-nonylphenol contained in the reaction solution in the absence of microorganisms
CB: The peak area of bisphenol B corresponding to CA
SA: The peak area of 4-nonylphenol not adsorbed by microorganisms
SB: The peak area of bisphenol B corresponding to SA
   (3) Atrazine Adsorption Effect of Various Microorganisms
   (a) Preparation of Sample

Samples were prepared in the same manner as in (1)(a) above.

(b) Preparation of Sample for Atrazine

100 mg of atrazine (manufactured by GL Sciences Inc.) was weighed in a 10 ml measuring flask using an electronic balance and dissolved in ethanol (manufactured by Wako Pure Chemicals Industries, Ltd., special grade) to obtain a final volume of precisely 10 ml. 3 ml of this solution was transferred to another measuring flask (50 ml) using a transfer pipette and diluted with ethanol to precisely 50 ml.

(c) Atrazine Adsorption by Microorganisms and Measurement of Residual Amount of Atrazine

0.2 ml of the ethanol solution of atrazine obtained in (b) above was added to the glass test tube containing 3.8 ml of the suspension of live bacteria or heat treated bacteria described in (a) above. The mixture was thoroughly stirred and allowed to stand in a water bath at 37° C. After 60 minutes, the cells were precipitated by centrifugation and 1 ml of the supernatant liquid was collected in a separate glass tube. 1 ml of dichloromethane was added, and the tube was vigorously agitated by a shaker. After 20 minutes, the dichloromethane layer was separated from the aqueous layer by centrifugation, and 0.2 ml of the dichloromethane layer was collected into another glass tube. The dichloromethane was evaporated to dryness and the residue was allowed to stand in a desiccator overnight. A sample obtained by carrying out the above procedure in the absence of microorganisms was used as a negative control.

0.2 ml of a 55:45 mixture of acetonitrile and 50 mM NH₄H₂PO₄ aqueous solution was added to the residue to redissolve the atrazine. Then, atrazine was quantitatively analyzed by HPLC under the following conditions.

(HPLC Measurement Conditions)

HPLC apparatus: Waters 2690 (manufactured by Waters Corp.)
Column: Symmetry C₁₈ 5 mm (manufactured by Waters Corp., 4.6×150 mm)
Column temperature: 37° C.
Flow rate: 1 ml/min.
Mobile phase: 55:45 mixture of acetonitrile (manufactured by Wako Pure Chemicals Industries Co., Ltd., for HPLC) and 50 mM NH₄H₂PO₄ aqueous solution
Detector: Waters 996 Photodiode Array Detector (manufactured by Waters Corp.)
Wavelength: 230 nm
Analytical time: 10 minutes
   (d) Calculation of the Amount of Atrazine Adsorption by Microorganisms

The amount of atrazine adsorbed by microorganisms was calculated from the amount of atrazine not absorbed by microorganisms determined in (c) above according to the following equation.
Y=X−(S/C)×X
Y: The amount of atrazine adsorbed by microorganisms
X: The amount of atrazine added to the reaction solution
S: The peak area of atrazine not adsorbed by microorganisms
C: The peak area of atrazine contained in the reaction solution in the absence of microorganisms
   (4) Bisphenol A Excretion Effect by Microorganisms in Rats
   (a) Preparation of Sample Diet and Experimental Groups of Animals

Sample diet compositions shown in Table 2 were prepared according to AIN-76 composition (Report of the American Institute of Nutrition Ad Hoc Committee on Standards for Nutritional Studies; J. Nutr., 107, 1340, 1977). Four experimental groups, each consisting of 8 animals, were used. Diet containing lyophilized live bacteria in the amount of 2.5%, 5%, or 10% was fed to each of the three groups, and diet containing no bacteria was given to the remaining one group (a control group). Each diet contained 15% of skim milk powder which was used for the protective agent when lyophilizing the bacteria. The diet compositions are shown in Table 2.

TABLE 2

| | | Bacteria administered | | |
|---|---|---|---|---|
| | Control | group (%) | | |
| Component | Group | 2.5% | 5% | 10% |
| Casein | 12.5 | 12.1 | 11.8 | 11.0 |
| DL-methionine | 0.3 | 0.3 | 0.3 | 0.3 |
| a-Cornstarch | 15.0 | 14.6 | 14.1 | 13.2 |
| Sucrose | 42.5 | 41.3 | 40.0 | 37.5 |
| Cellulose fiber | 5.0 | 4.9 | 4.7 | 4.4 |
| Corn oil | 5.0 | 4.9 | 4.7 | 4.4 |
| AIN-76 mineral added | 3.5 | 3.4 | 3.3 | 3.1 |
| AIN-76 vitamin added | 1.0 | 1.0 | 0.9 | 0.9 |
| Choline bitartrate | 0.2 | 0.2 | 0.2 | 0.2 |
| Defatted skim milk | 15.0 | 15.0 | 15.0 | 15.0 |
| Lyophilized live bacteria | 0.0 | 2.5 | 5.0 | 10.0 |

(b) Animals and Feeding Method

Fisher 344 female rats (age: 10 weeks, supplied by Clea Japan, Inc.) were bred to be acclimated (MF diet manufactured by Oriental Yeast Co., Ltd. was freely given.). After one week, the rats were classified into seven groups according to the weight. The test diet was administered for 12 days during which the animals were provided as much diet and water as they like. A cycle of 12-hour light condition and 12-hour dark condition at room temperature of 25° C. and a relative humidity of 55% was applied to breeding.

(c) Method of Administering Bisphenol A and Collecting Faeces

Five days after start of feeding the test diet, the animals were fasted over night. On the following day, 10 g of a test sample diet containing 100 μg of bisphenol A was administered. The total amount of faeces was collected and lyophilized for seven days after start of bisphenol A administration.

(d) Extraction of Bisphenol A from Feces

Bisphenol A was extracted from the feces according to the method described in MANUAL FOR INVESTIGATION OF ENDOCRINE DISRUPTING CHEMICALS 1N SEWERAGE (Japan Institute of Wastewater Engineering Technology) and ANALYSIS OF ENDOCRINE DISRUPTING CHEMICALS (ENVIRONMENT HORMONES) (III) (GL Sciences, Inc.).

An organic solvent for the pesticide residue examination was used for the extraction of bisphenol A. 0.5 g of lyophilized feces were put into a glass tube and 1 μg of bisphenol A-d16 (surrogate substance) and 10 ml of methanol was added. The mixture was vigorously stirred (200 stroke/min). The supernatant liquid obtained by centrifugation (2,000 rpm, 20 minutes) was collected in a separate glass tube. 10 ml of methanol was added to the residue. The mixture was vigorously stirred and combined with the previously obtained supernatant liquid. The supernatant liquid (about 20 ml) was concentrated to about 5 ml. After the addition of 0.125 ml of purified water and 2.5 ml of n-hexane, the mixture was vigorously stirred (200 stroke/min). After 10 minutes, n-hexane layer eliminated and then 2.5 ml of fresh n-hexane was added to the methanol layer and the mixture was vigorously stirred (200 stroke/min). After 10 minutes, the methanol layer was concentrated to about 1 ml and the resulting concentrate was adjusted to pH 3 by the addition of 0.125 N HCl aqueous solution.

4 ml of 3.75% brine and 5 ml of dichloromethane were added to the solution, and the mixture was vigorously stirred (200 stroke/min). After 10 minutes, the lower dichloromethane layer was transferred to a separate glass tube. 5 ml of fresh dichloromethane was added to the upper layer and the mixture was vigorously stirred (200 stroke/min). After 10 minutes, the dichloromethane layer was combined with the previously obtained dichloromethane layer, and the mixture was concentrated to about 5 ml. 1.5 g of anhydrous sodium sulfate was added to the concentrate to dehydrate. The solution was loaded onto a silica gel column (40 mm×12 mm) and 10 ml of n-hexane was fed, while discharging the eluate. Next, 10 ml of acetone was fed while collecting the eluate. The resulting eluate was dried to solidify, 0.26 ml of dichloromethane was added to the residue to redissolve the bisphenol A.

BSTFA was added to convert the phenolic group into a trimethylsilyl group. The mixture was allowed to stand for one hour at room temperature. After the trimethylsilylation reaction, 0.1 ml of a solution of phenanthrene-d10 (the internal standard substance) in dichloromethane (10 μg/ml) was added and bisphenol A was quantitatively analyzed by gas chromatography/mass spectrometer (GC/MS) under the following conditions.

(e) GC/MS Measuring Conditions

The GC/MS was measured in the same manner as in (2) (d) above.

(f) Quantitative Analysis of Bisphenol A

The detected amount of bisphenol A was determined using a calibration curve from the ratio of the peak area of the trimethylsilylation product of bisphenol A and that of the bisphenol A-d16 determined by the GC/MS measurement. The bisphenol A concentration was then calculated from the detected amount, the amount of sample injected into GC/MS apparatus, the amount of feces used in the test, the concentration magnification, etc. using the following formula.
Y=D×(L×1000/I)/F
Y: The amount of bisphenol A adsorbed by microorganisms (μg/g)
D: Amount detected by the GC/MS (μg)
L: Final liquid amount of the sample (ml)
I: Amount sample injected to GC/MS (μl)
F: Amount of feces used in the test (g)
   (g) Measurement of the Number of Live Bacteria in the Cecum Contents

The cecum was collected from the rats to which the live bacteria were administered for 12 days according to (a) above under pentobarbital anesthesia, and stored in ice. The number of live bacteria in the cecum contents was measured according to the method of Yuki et al. (Norikatsu YUKI, Yukiko SAKAITANI, Yoko TAGAMI, and Masami MOROTOMI, Digestive Tract Remaining Properties Of Lactobacillus casei Strain Shirota In Fermented Milk, Journal of Nutrition Food 2, 1-6, 1999) or the method of Asahara et al. (Takashi ASAHARA, Kensuke SHIMIZU, Yuji OHASHI, Takahiro MATSUKI, Kazumasa MATSUMOTO, Toshihiko TAKADA, Norikatsu YUKI, Hiroo TAKAYAMA, and Ryuichiro TANAKA, The Effects of Bifidobacteria-Fermented Milk on Human Urinary Mutagenicity, Which Increases Following Ingestion of Cooked Ground Beef, Journal of Intestinal Microbiology, 12, 89-96, 1999).

(h) Statistical Analysis

Statistical analysis of the resulting data was carried out according to the multiple comparison procedure of Dunnett.

### Example 1

Adsorption of Bisphenol A by Lactobacillus casei YIT 9029 (1)

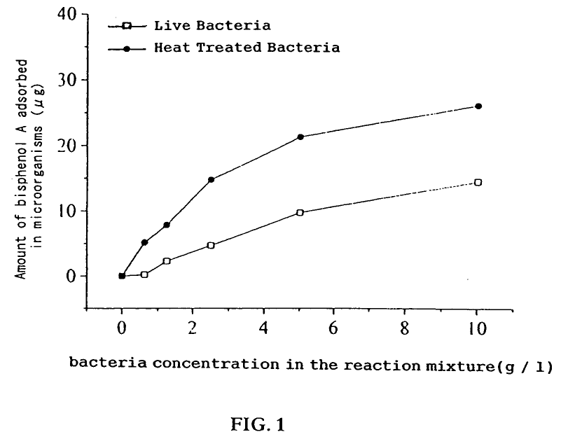

Live bacteria and heat treated bacteria of Lactobacillus casei YIT 9029 were obtained according the method of sample preparation described in (1) (a) above. The relation between the microorganism concentration and the amount of bisphenol A adsorbed was investigated using these samples. Test samples of live bacteria and heat treated bacteria of Lactobacillus casei YIT 9029 with various concentrations (0, 0.625, 1.25, 2.5, 5, and 10 g/l) were prepared. 40 μg of bisphenol A was added to these samples and reacted, following which the amount of bisphenol A remaining in the supernatant liquid was analyzed by gas chromatography to determine the amount of bisphenol A adsorbed. The results are shown in FIG. 1.

(Results)

As shown in FIG. 1, the amount of bisphenol A adsorbed in Lactobacillus casei YIT 9029 increased in accordance with the increase in the cell concentration in the reaction solution. The adsorption effect was seen in both live bacteria and heat treated bacteria. The heat treated bacteria were confirmed to adsorb a larger amount of bisphenol A than live bacteria. The adsorption effect of both the live bacteria and heat treated bacteria of the present invention was thus demonstrated.

Therefore, it was confirmed that the foods and drinks manufactured using these microorganisms does not necessarily require to include live bacteria, and such foods and drinks may be manufactured under wide spectrum of conditions in terms of temperature, pH, and the like.

### Example 2

Adsorption of Bisphenol A by Lactobacillus casei YIT 9029 (2)

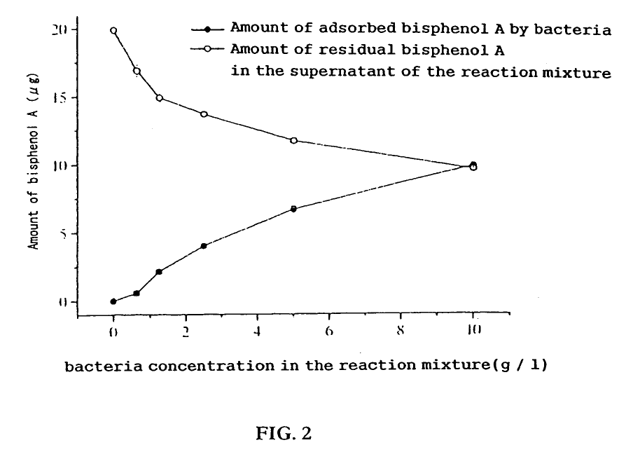

The relation between the bacteria concentration, the amount of adsorbed bisphenol A, and the amount of remaining bisphenol A was investigated using live bacteria of Lactobacillus casei YIT 9029 as microorganism. Test samples of live bacteria of Lactobacillus casei YIT 9029 with various concentrations (0, 0.625, 1.25, 2.5, 5, and 10 g/l) were prepared. 20 μg of bisphenol A was added to these samples. After the reaction, the amount of bisphenol A adsorbed by bacteria and the amount of remaining bisphenol A were determined by the quantitative analysis according to the GC/MS method and the gas chromatography method respectively described in (1)(c) and (1)(e) above and according to the calculation described in (1)(d) and (f) above. The results are shown in FIG. 2.

(Results)

As shown in the FIG. 2, the amount of bisphenol A adsorbed in the Lactobacillus casei YIT 9029 increased according to the increase in the concentration of the microorganism in the reaction solution. In contrast, the amount of bisphenol A remaining in the supernatant of the reaction solution after centrifugation decreased according to the increase in the concentration of the microorganism. In this instance, the total sum of the adsorbed bisphenol A and the bisphenol A remaining in the supernatant was equivalent to the amount of bisphenol A (20 μg) added to the reaction solution.

This indicates that all bisphenol A not adsorbed by bacteria is present in the supernatant obtained by centrifugation. Therefore, the amount of bisphenol A adsorbed in bacteria calculated from the bisphenol A remaining in the supernatant after centrifugation is equivalent to the amount of bisphenol A actually adsorbed by the bacteria.

### Example 3

Adsorption of Bisphenol A by Lactobacillus casei YIT 9029 (3)

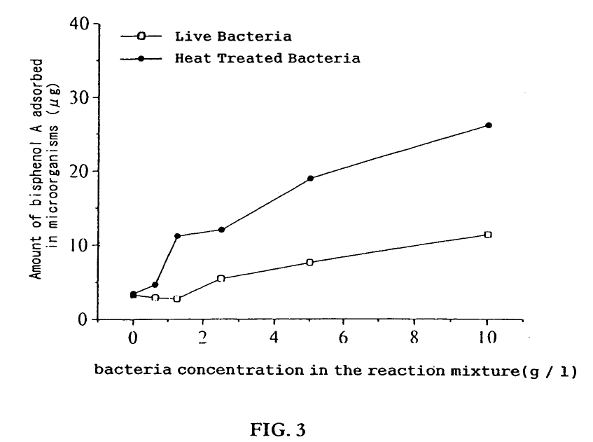

The relation between the concentration microorganism or diet and the amount of bisphenol A adsorbed was investigated using live bacteria and heat treated bacteria of Lactobacillus casei YIT 9029 as the microorganism and a diet for animal tests (“F-2” manufactured by Funabashi Farm Company). Test samples of live bacteria or heat treated bacteria of Lactobacillus casei YIT 9029, of the diet with various concentrations (0, 0.625, 1.25, 2.5. 5, and 10 g/l) were prepared. 40 μg of bisphenol A was added to these samples and reacted, following which the amount of bisphenol A remaining in the supernatant liquid was analyzed by gas chromatography to determine the amount of bisphenol A adsorbed. The results are shown in FIG. 3.

(Results)

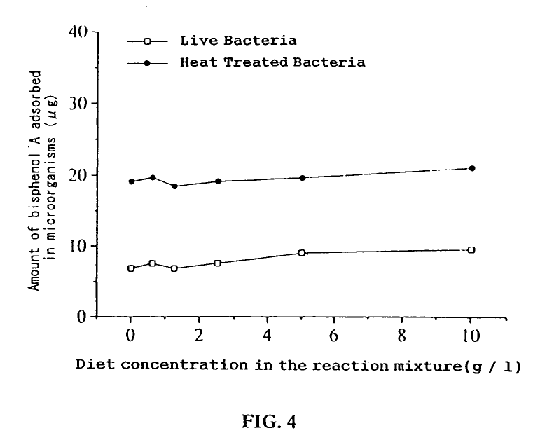

As shown in FIG. 3, the amount of bisphenol A adsorbed in Lactobacillus casei YIT 9029 increased in accordance with the increase in the cell concentration in the reaction solution in a certain range of diet concentration. On the other hand, as shown in FIG. 4, the amount of bisphenol A adsorbed by Lactobacillus casei YIT 9029 cells of a certain concentration range was not affected by the diet concentration in the reaction solution.

These results suggest the capability of Lactobacillus casei YIT 9029 cells to selectively adsorb bisphenol A in the digestive tract of humans and animals without being affected by the presence or absence of diet.

### Example 4

Adsorption of bisphenol A by Lactobacillus casei YIT 9029 (4)

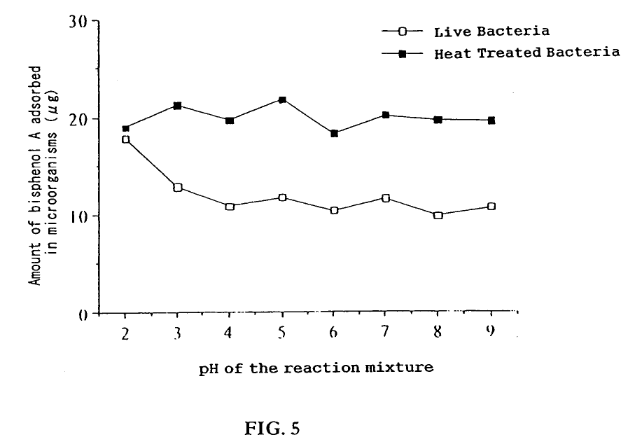

The relation between the pH and and the amount of bisphenol A adsorbed was investigated using live bacteria and heat treated bacteria of Lactobacillus casei YIT 9029 as the microorganism. 20 mg of Lactobacillus casei YIT 9029 cells and 40 μg of bisphenol A dissolved in 4 ml of a buffer solution was heated to 37° C., and the amount of adsorption was measured after 60 minutes. Sample solutions with a pH of 2-9 were prepared. Buffer solutions with a pH between 2 and 4 were prepared by adding hydrochloric acid to a 20 mM KH₂PO₄ aqueous solution. Buffer solutions with a pH between 5 and 8 were prepared by mixing a 20 mM KH₂PO₄ aqueous solution and a 20 mM K₂HPO₄ aqueous solution, and a buffer solution with a pH of 9 was prepared by using a 20 mM K₂HPO₄ aqueous solution. The results are shown in FIG. 5.

(Results)

As shown in FIG. 5, when heat treated bacteria Lactobacillus casei YIT 9029 were used, the amount of bisphenol A adsorbed was almost the same irrespective of the pH of the reaction solution. In contrast, live bacteria adsorbed a larger amount of bisphenol A in an acidic range (pH 2). Heated cells adsorbed a larger amount of bisphenol A than live bacteria in all pH range.

These results confirm capability of the microorganisms to adsorb bisphenol A in all parts of digestive tract with a differing pH from the esophagus to the anus. In addition, the effect is exhibited by using either live bacteria or dead bacteria.

### Example 5

Adsorption of Bisphenol A by Lactobacillus casei YIT 9029 (5)

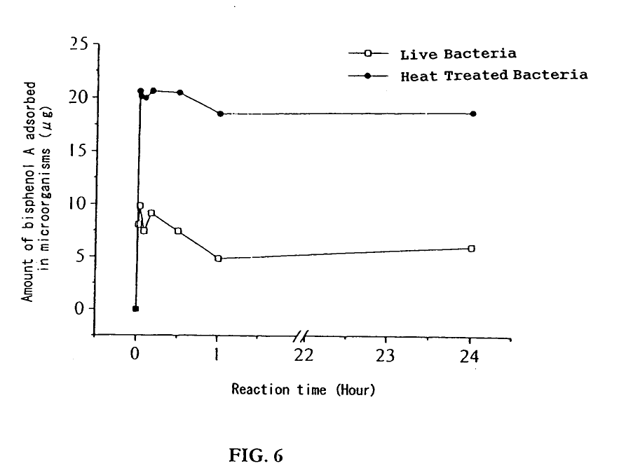

The relation between the reaction time and the amount of bisphenol A adsorbed was investigated using live bacteria and heat treated bacteria of Lactobacillus casei YIT 9029 as the microorganism. 20 mg of Lactobacillus casei YIT 9029 cells and 40 μg of bisphenol A suspended in 4 ml of a buffer solution was heated to 37° C., and the amount of adsorption was measured after 1 minute, 2 minutes, 5 minutes, 10 minutes, 30 minutes, 60 minutes, and 1440 minutes (24 hours). The results are shown in FIG. 6.

(Results)

As shown in FIG. 6, in the both tests using live bacteria or heat treated bacteria, the amount of bisphenol A adsorbed reached the maximum immediately after the start of reaction, and the effect of adsorption was maintained throughout the entire test of 24 hours (1440 minutes).

These results suggest that the microorganisms which have adsorbed bisphenol A can be transported in the digestive tract to the anus without releasing the bisphenol A, and excreted as excrements.

### Example 6

Bisphenol A Adsorption by Various Microorganisms

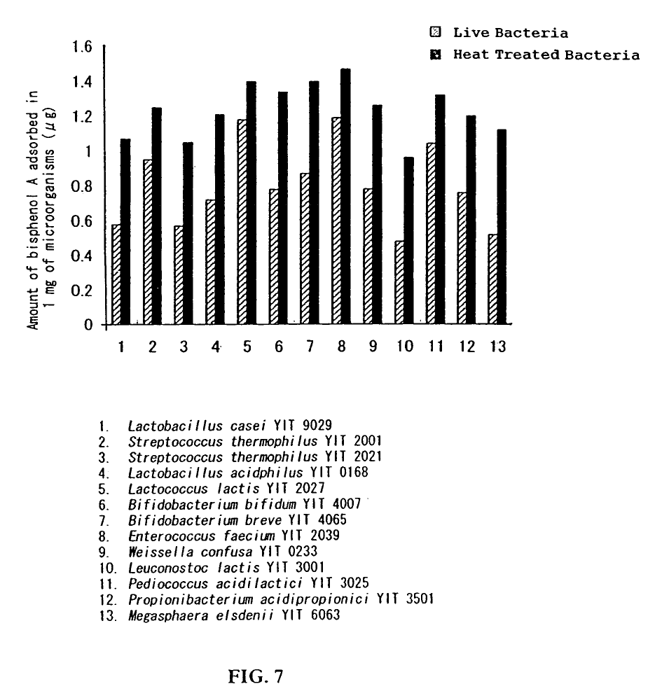

20 mg of live bacteria or heat treated bacteria and 40 μg of bisphenol A suspended in 4 ml of a buffer solution was allowed to stand at a temperature of 37° C. After 60 minutes, the amount of bisphenol A remaining in the supernatant after centrifugation was measured to determine the amount of bisphenol A adsorbed by the bacteria. An amount of bisphenol A (μg) adsorbed by 1 mg of cells was used to indicate the unit amount of bisphenol A adsorbed by microorganisms. The results are shown in FIG. 7.

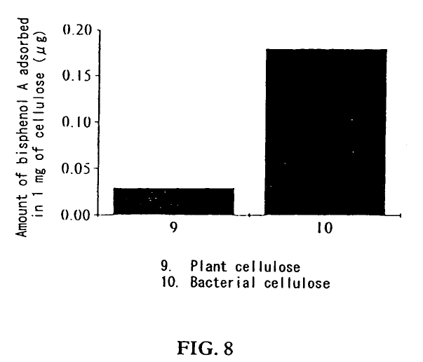

For comparison, capability of celluloses, which are said to adsorb endocrine-disrupting chemicals, to adsorb bisphenol A was examined. Plant cellulose (manufactured by Toyo Roshi Co., Ltd.) and bacterial cellulose (cellulose produced by Acetobactor pasteuria YIT 6109) having more fine fiber structure than the plant cellulose, both used as food fibers in animal tests, were used as the cellulose. The results are shown in FIG. 8.

(Results)

As shown in FIG. 7, all microorganisms of the present invention adsorbed bisphenol A, although the amount of adsorption differed according to the type of microorganisms. Both live bacteria and dead bacteria of any microorganisms adsorbed a larger amount of bisphenol A.

On the other hand, as shown in FIG. 8, plant celluloses adsorbed almost no bisphenol A. Bacterial cellulose having more fine fiber structure than plant cellulose also adsorbed bisphenol A, but the effect of bisphenol A adsorption was significantly smaller than the microorganisms of the present invention.

These results suggest that the effect of adsorbing bisphenol A is not necessarily seen in all food fibers and microorganisms on the across-the-board basis, but is inherent to the enterobacteria of the present invention.

### Example 7

4-Nonylphenol Adsorption Test Using Various Microorganisms

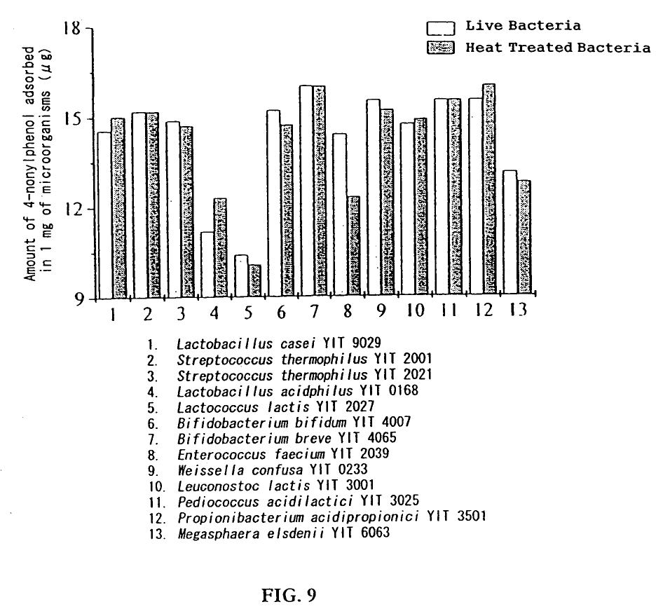

2.5 mg of live bacteria and 40 μg of 4-nonylphenol were suspended in 4 ml of a buffer solution and allowed to stand at 37° C. The amount of the cells adsorbed was measured after 60 minutes. The amount of 4-nonylphenol adsorbed was indicated by the amount of 4-nonylphenol (μg) absorbed by 1 mg of the cells. The results are shown in FIG. 9.

(Results)

As shown in FIG. 9, all microorganisms of the present invention adsorbed 4-nonylphenol, although the amount of adsorption differed according to the type of microorganisms. Both live bacteria and dead bacteria of any microorganisms adsorbed a larger amount of 4-nonylphenol.

### Example 8

Atrazine Adsorption Test Using Various Microorganisms

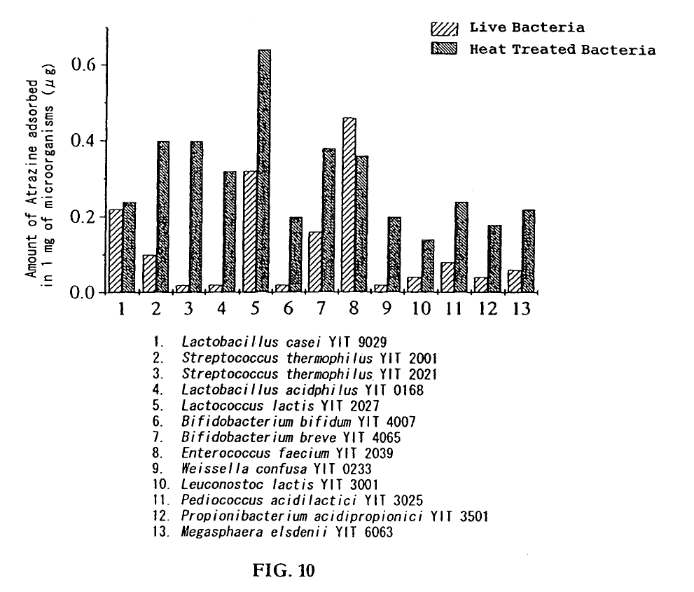

20 mg of live bacteria or heat treated bacteria and 40 μg of atrazine were suspended in 4 ml of a buffer solution and allowed to stand at 37° C. The amount of the cells adsorbed by atrazine was measured after 60 minutes. An amount of atrazine (μg) adsorbed by 1 mg of cells was used to indicate the unit amount of atrazine adsorbed by microorganisms. The results are shown in FIG. 10.

(Results)

As shown in FIG. 10, all microorganisms of the present invention adsorbed atrazine, although the amount of adsorption differed according to the type of microorganisms. Both live bacteria and dead bacteria of any microorganisms adsorbed a larger amount of atrazine.

### Example 9

Adsorption of Bisphenol A by Lactobacillus casei YIT 9029 or Bifidobacterium breve YIT 4065 in Rat Intestinal Tracts (1)

The relation between the amount of bacteria administered and the amount of bisphenol A excreted was examined using Lactobacillus casei YIT 9029 or Bifidobacterium breve YIT 4065 as microorganism. Animal diets prepared by adding lyophilized bacteria of Lactobacillus casei YIT 9029 or Bifidobacterium breve YIT 4065 to final concentrations of 0%, 2.5%, 5%, and 10% were used as test samples. For comparison, diet prepared by adding food cellulose (manufactured by Toyo Roshi Co., Ltd.) to a final concentration of 10% was used. According to the procedure described in (4)(c), the animals were fasted over night five days after start of feeding the test diet, and 10 g of a test sample diet containing 100 μg of bisphenol A was administered.

The total amount of feces was collected for seven days after start of bisphenol A administration to measure the weight of feces, the amount of bisphenol A in the feces, the food consumption, and the body weight increase. In addition, the number of live bacteria in the cecum contents of rats was measured on the seventh day after start of sample diet administration.

(Results)

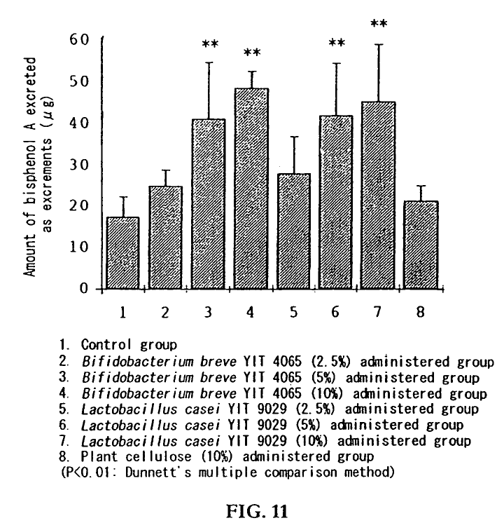

As shown in FIG. 11, the amount of bisphenol A excreted in feces by rats in the control group during seven days was 17 μg, only 17% of bisphenol A administered (100 μg). On the other hand, in rats to which Lactobacillus casei YIT 9029 or Bifidobacterium breve YIT 4065 was administered, the amount of bisphenol A excreted in feces increased according to the increase in the amount of bacteria administered. It was confirmed that 45-48% of bisphenol A administered was excreted when the diet containing 10% of these bacteria was fed to the animals. There were no difference in the body weight and the amount of diet among the tested groups during the test period.

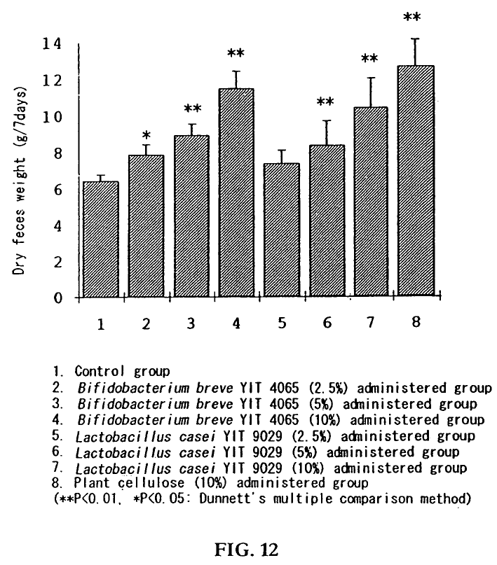

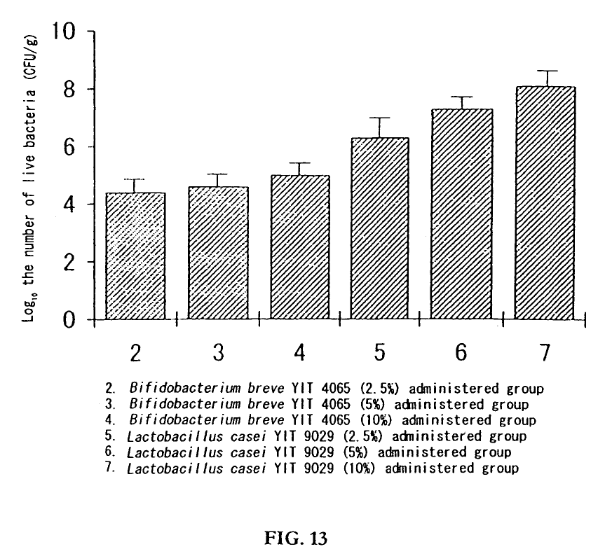

On the other hand, as shown in FIG. 12, the amount of feces excreted during the first seven days of bisphenol A administration increased according to the increase in the amount of bacteria administered. In addition, as shown in FIG. 13, the number of live bacteria in the cecum contents of rats on the seventh day of bisphenol A administration also increased according to the increase in the amount of bacteria administered. These results indicate that live bacteria in the intestine increases by the administration of bacteria and these results in accelerated excretion of feces.

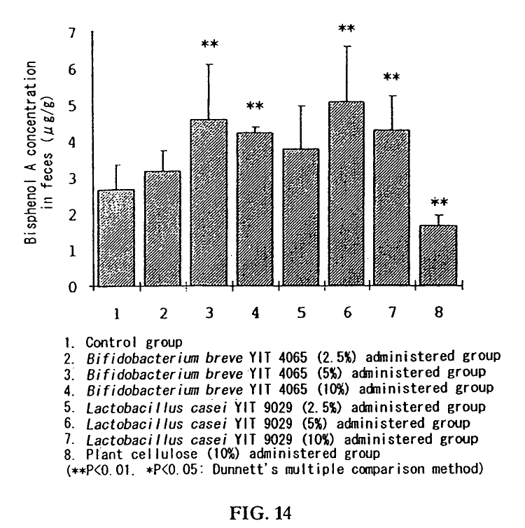

Furthermore, as shown in FIG. 14, the concentration of bisphenol A excreted in feces by rats varied among the groups, and all groups to which the bacteria were administered exhibited a significant increase as compared with the control group and the group to which plant cellulose was administered.

All these results show that Lactobacillus casei YIT 9029 and Bifidobacterium breve YIT 4065 can suppress absorption of bisphenol A taken together with diet from the intestinal tract and accelerate the excretion in feces. The results also indicate that the increase in the excretion of bisphenol A in feces by the administration of bacteria is caused not only by the increase of feces excretion due to administration of bacteria, but also by the increased adsorption of the substance by the bacteria.

### Example 10

Preparation Various Food Compositions

Various food compositions were prepared using the microorganisms of the present invention. The formulations are shown below, which should not be construed as limiting the present invention.

(1) Health Supplement (Tablets)

A composition with the following formulation was made into tablets.
(Component) Amount (wt %)
Dried enterobacterial cells¹⁾ 10
Plat extract powder 30
Royal jelly powder 5
Collagen powder 5
Lactose 25
Corn starch 20
Hydroxypropylcellulose 4
Magnesium stearate 1
   1) Prepared by freeze-drying the live bacteria or heat treated bacteria of Lactobacillus casei YIT 9029 obtained in Example 1.
   (2) Health drink

A health promotion drink with the following formulation was prepared.
(Component) Amount (wt %)
Dried enterobacterial cells¹⁾ 5
Honey 15
Citric acid 0.1
dl-malic acid 0.1
Plat extract liquid (cinnamon) 20
D-Sorbitol solution (70%) 10
Sodium benzoate 0.05
Perfume Appropriate amount
Purified water Balance to make the total 100
   1) Prepared by freeze-drying the live bacteria or heat treated bacteria of Lactobacillus casei YIT 9029 obtained in Example 1.
   (3) Fruit Juice

Fruit juice with the following formulation was prepared.
(Component) Amount (wt %)
Dried enterobacterial cells¹⁾ 5
Liquid glucose 33
Grapefruit fruit juice 60
Perfume Appropriate amount
sourness additive Appropriate amount
   1) Prepared by freeze-drying the live bacteria or heat treated bacteria of Lactobacillus casei YIT 9029 obtained in Example 1.
   (4) Fermented Milk

Fermented milk was prepared as follows.

(A) Fermented Milk Produced from One Microorganism Strain

Fermented milk was prepared by sterilizing a mixture of 10% skim milk powder and 5% glucose, and inoculating an enterobacterium. Eight types of fermented milk were prepared by using any one of the following enterobacteria: Lactobacillus casei YIT 9029, Lactobacillus acidophilus YIT 0168, Bifidobacterium breve YIT4065, Bifidobacterium bifidum YIT4007,—Lactococcus lactis YIT2027, Streptococcus thermophilus YIT2001, Streptococcus thermophilus YIT2021, and Enterococcus faecium YIT2039. The amount of cells contained in the resulting fermented milks was between 0.1 g/l and 10 g/l (the number of live bacteria: about 4×10⁸ to 4×10¹⁰ cells/ml) All of the fermented milks had a good aroma and were highly tasty.

(B) Fermented Milk Produced from Combinations of Microorganism Strains

Ten types of fermented milk were prepared by sterilizing a mixture of 10% skim milk powder and 5% glucose, and inoculating the following combinations of enterobacteria.
Combination 1: Lactobacillus acidophilus YIT0168 and Lactobacillus casei YIT9029.
Combination 2: Bifidobacterium breve YIT4065 and Lactobacillus casei YIT9029.
Combination 3: Bifidobacterium bifidum YIT4007 and Lactobacillus casei YIT9029.
Combination 4: Lactococcus lactis YIT2027 and Lactobacillus casei YIT9029.
Combination 5: Streptococcus thermophilus YIT2001 and Lactobacillus casei YIT9029.
Combination 6: Streptococcus thermophilus YIT2021 and Lactobacillus casei YIT9029.
Combination 7: Enterococcus faecium YIT2039 and Lactobacillus casei YIT9029.
Combination 8: Bifidobacterium breve YIT4065, and Bifidobacterium bifidum YIT4007, and Lactobacillus acidophilus YIT0168.
Combination 9: Streptococcus thermophilus YIT2021, Bifidobacterium breve YIT4065, and Lactobacillus acidophilus YIT0168.
Combination 10: Streptococcus thermophilus YIT2001, Bifidobacterium breve YIT4065, and Lactococcus lactis YIT2027.

The total amount of cells contained in the resulting fermented milks was between 0.1 g/l and 10 g/l (the number of live bacteria: about 4×10⁸ to 4×10¹⁰ cells/ml). All these fermented milks had a good aroma and were highly tasty.

### INDUSTRIAL APPLICABILITY

As described above, live bacteria or dead bacteria of enterobacteria or the constituents thereof can adsorb endocrine-disrupting chemicals which can adsorb and eliminate orally taken endocrine-disrupting chemicals such as bisphenol A, 4-nonylphenol, and atrazine in the intestinal tract of humans or animals, and not only can suppress in vivo absorption of such chemicals, but also accelerate excretion of such in vivo-absorbed chemicals. Thus, the cells and the constituents are extremely useful for preventing human organs from being polluted with endocrine-disrupting chemicals.

The enterobacteria have been used for the production of foods such as lactic acid bacteria beverage, yogurt, and the like, and are extremely safe microorganisms free from pathogenicity.

Therefore, the adsorbent for endocrine-disrupting chemicals containing the above cells of enterobacteria of the present invention not only can be used as a medicine or oral administration, but also added to foods and taken in daily life. Thus, the adsorbent of the present invention is very useful for protecting humans from being polluted with endocrine-disrupting chemicals and maintaining health of humans.

1-9. (canceled)

10. A method for reducing the absorption of at least one endocrine-disrupting chemical comprising administering to a subject in need thereof an amount of enterobacteria or an amount of an enterobacteria fraction effective to reduce the absorption of said endocrine-disrupting chemical.

11. The method of claim 10, wherein the endocrine-disrupting chemical is selected from the group consisting of at least one bisphenol, alkylphenol and triazine.

12. The method of claim 10, wherein said subject is a non-human animal.

13. The method of claim 10, wherein said subject is human.

14. The method of claim 10, wherein said enterobacteria is at least one selected from the group consisting of Lactobacillus, Bifidobacterium, Lactococcus and Streptococcus.

15. The method of claim 10, wherein said enterobacteria is at least one selected from the group consisting of Enterococcus, Weissella, Leuconostoc, Tetragenococcus, Propionibacterium, Bacteroides, Clostridium, Eubacterium, Prevotella, Pediococcus, and Megasphaera.

16. The method of claim 10, comprising administering isolated live enterobacteria.

17. The method of claim 10, comprising administering isolated dead enterobacteria.

18. The method of claim 10, comprising administering an isolated protoplasmic fraction of an enterobacterium.

19. The method of claim 10, comprising administering an isolated soluble fraction of an enterobacterium.

20. The method of claim 10, comprising administering an isolated cytoplasmic membrane fraction of an enterobacterium.

21. The method of claim 10, comprising administering an isolated cell wall fraction of an enterobacterium.

22. The method of claim 10, comprising administering a pulverized enterobacterium.

23. The method of claim 10, comprising administering a dose of enterobacteria or an enterobacteria fraction ranging from 10 mg to 30 g/day.

24. The method of claim 10, comprising administering a dose of enterobacteria or an enterobacteria fraction ranging from 1 to 5 g/day.

25. The method of claim 10, wherein an isolated enterobacteria or enterobacteria fraction is administered.

26. The method of claim 10, wherein a food supplemented with the isolated enterobacteria or enterobacteria fraction is administered.

27. The method of claim 10, wherein a non-fermented food containing the isolated enterobacteria or enterobacteria fraction is administered.

28. The method of claim 10, wherein a food containing the isolated enterobacteria or enterobacteria fraction is administered and said food is selected from the group consisting of fruit juice, soup, rice cracker and cookie.

29. The method of claim 10, wherein said enterobacteria or said enterobacteria fraction accelerates the excretion of the endocrine-disrupting chemical.
